(19) United States
(12) Patent Application Publication
(10) Pub. No.: US 2005/0265952 A1
(43) Pub. Date: Dec. 1, 2005

(54) Thickener for personal care compositions

(76) Inventor: Bharat B. Desai, Spartanburg, SC (US)

Correspondence Address: Isaac A. Angres, Suite 301, 2001 Jefferson Davis Highway, Arlington, VA, 22202, US

(21) Appl. No.: 11/135,378

(22) Filed: May 24, 2005

Related U.S. Application Data
(60) Provisional application No. 60/573,836, filed on May 25, 2004.

(51) Int. Cl. A61K 7/075; A61K 7/08

(52) U.S. Cl. 424/70.31

(57) ABSTRACT

The present invention provides a cosmetic product comprising cosmetically acceptable ingredients in a liquid carrier, and a thickener of the formula 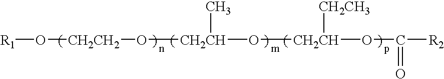
wherein R₁ is a saturated or unsaturated C₈-C₄₀ group; R₂ is a saturated or unsaturated C₈-C₂₄ group, n=1-200, m=0-200, and p=0-200.

### FIELD OF INVENTION

This invention relates to the technical sector of cosmetic, dermatological, pharmaceutical or veterinary, and detergent compounds comprising at least one thickening and/or emulsifying agent for aqueous and non-aqueous media.

The present invention features novel personal products or detergent compositions containing novel thickening agents. The instant invention also generally relates to aqueous surfactant solutions containing special thickeners. This invention further relates to thickeners or viscosifers for increasing the viscosity of aqueous and non-aqueous compositions. In particular, this invention relates to thickeners which are easily incorporated into aqueous-based cosmetics and other personal care compositions. The invention also relates to improved thickeners for cosmetic compositions, particularly those in lotion and cream form and other desirable aesthetic forms.

The instant invention is also directed to compositions specifically tailored for use as thickeners/stabilizers for products for topical application such as personal care products for application to the skin or hair, or topically administrable pharmaceutical preparations, where the formulated products are of an anionic or nonionic character.

The present invention also provides mild liquid personal cleanser compositions incorporating thickeners based on fatty acid esters of fatty alcohol alkoxylates. More specifically, the present invention relates to mild, high lathering compositions with modified rinse feel attributes targeted for all-body use as shower gels.

The present invention further relates to thickeners derived from alkoxylated fatty alcohols which have been esterified with fatty acids.

This invention also describes aqueous surfactant solutions containing fatty acid ester of fatty alcohol alkoxylates as thickeners, to the use of fatty acid esters of fatty alcohol alkoxylates as thickeners in aqueous surfactant solutions and to processes for the production of fatty acid esters of fatty alcohol alkoxylates.

### BACKGROUND OF THE INVENTION

Many aqueous and non-aqueous systems require thickeners in order to be useful for various types of applications. Such aqueous-based systems as cosmetics, cleansing products, protective coatings for paper and metal, printing inks, and latex paints all require the incorporation of thickeners in order to have the proper rheological characteristics for their particular uses. Many substances useful as thickeners are known in the art. These include natural polymers such as alginates, casein and synthetic materials such as cellulose derivatives, acrylic polymers, and polyurethane polymers. Some thickeners for use in personal care products such as shampoos, facial cleaners, liquid hand soaps, and the like require prolonged mixing periods and high temperatures to be incorporated into the products to be thickened. In some instances, the effectiveness of some thickeners has been found to be influenced by the components in the compositions to be thickened.

Also, aqueous cosmetic compositions often require thickeners to achieve an aesthetically pleasing viscosity. Fluids that flow with a watery consistency too rapidly run off the treated skin areas. For a cosmetic to be effective, it often must have substantivity. Thickeners provide this substantivity. Furthermore, low viscosity formulas which may be skin effective nevertheless through their wateriness signal ineffectiveness to the consumer. Products of watery consistency are also aesthetically displeasing to consumers with expectations of rich and creamy products.

Personal care compositions such as cleansing products must also satisfy a number of criteria to be acceptable to consumers. These criteria include cleansing power, skin feel, mildness to the skin, hair, and occular mucosae. Ideal personal cleansers should gently cleanse the skin or hair, causing little or no irritation and without leaving the skin overly dry or taut after frequent use.

Additionally, aqueous surfactant solutions, particularly those used in the field of personal care as hair shampoos, foam baths, shower baths, hand washing pastes and the like, mostly contain anionic surfactants, for example alkyl ether sulfates. In order to stabilize these clear or disperse systems and to make them easier for the user to handle, thickeners are normally added to the surfactant solutions. Various inorganic and organic compounds used to increase the viscosity of anionic surfactant solutions are known in the art. Water-soluble electrolyte salts, typically sodium chloride, are generally used as inorganic thickeners. Examples of organic thickeners are fatty acid alkanolamides, polyethylene glycol difatty acid esters and a number of water-soluble polymers. In most cases, the required viscosity of the surfactant solution can be adjusted with inorganic electrolyte salts alone, but only by using large quantities. Accordingly, organic thickeners are generally used in addition to the inorganic salts although, in some cases, they are have a number of disadvantages. For example, surfactant solutions thickened with polyethylene glycol fatty acid diesters often show inadequate viscosity stability in storage while water-soluble polymers show unwanted slimy flow behavior with a tendency to become stringy in the thickened surfactant solutions.

The formulation of shampoos necessitates a balance between two conflicting physical properties. On the one hand, consumer conception demands a higher viscosity product, while on the other hand, economics require a product low in solid content. Typically, today's shampoos have a solid content in the range of 6 to 18 percent. Specific viscosity modifying agents used in shampoo compositions have included PPG-5-Ceteth-20, and particularly, PEG 150 Distearate These agents distinguish themselves from other classes of cosmetic thickening agents by their low reactivity, low toxicity and usefulness over a broad pH range. These thickening agents are, however, linear in secondary structure and are able to fold over upon themselves thereby limiting their tendency to behave in a manner characteristic of long chain polymers. In addition, PEG 6000 Distearate is difficult to manufacture, often forming molecules having a different structure from batch to batch, i.e., containing free stearic acid, distearates and monostearates.

While different thickeners have been disclosed in various applications, there still is the desire to find a thickener which provides suitable thickening as well as other rheological properties needed for personal care applications. This invention provides a thickener-rheology modifier which significantly improves the thickening properties of personal care products and makes the products aesthetically acceptable for personal care use.

### OBJECTS OF THE INVENTION

It is a first object of the present invention to provide personal care products incorporating novel thickeners.

It is another object of the instant invention to provide cosmetic products incorporating novel thickeners.

A further object of the invention is to provide novel cosmetic products incorporating thickeners based on fatty acid esters of fatty alcohol alkoxylates.

A still further object of the invention is to provide novel shampoos incorporating thickeners based on fatty acid esters of fatty alcohol alkoxylates.

An additional object of the invention is to provide novel cosmetic products incorporating thickeners based on fatty acid esters of fatty alcohol ethoxylates.

A still further object of the invention is to provide novel aqueous surfactant solutions incorporating thickeners based on fatty acid esters of fatty alcohol alkoxylates.

An additional object of the present invention is to provide fatty acid esters of fatty alcohol alkoxylates.

These and other objects of the present invention will more readily become apparent from the description and examples which follow.

### SUMMARY OF THE INVENTION

The present invention relates to a personal care product comprising appropriate ingredients in a liquid carrier, wherein said personal care product includes an effective amount to thicken said product of a thickener of the formula 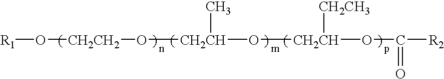
wherein R₁ is a saturated or unsaturated C₈-C₄₀ group; R₂ is a saturated or unsaturated C₈-C₂₄ group, n=1-200, m=0-200, and p=0-200.

The invention is also directed to a cosmetic product comprising cosmetically acceptable ingredients in a liquid carrier, said product including an effective amount to thicken said product of a thickener of the formula 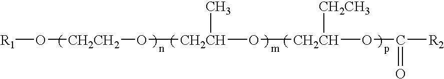
wherein R₁ is a saturated or unsaturated C₈-C₄₀ group; R₂ is a saturated or unsaturated C₈-C₂₄ group, n=1-200, m=0-200, and p=0-200.

The instant invention also relates to an aqueous composition comprising a surfactant selected from the group consisting of anionic, cationic, non-ionic, switterionic, and amphoteric surfactants and from 0.5 to 5% by weight of a thickener of the formula: 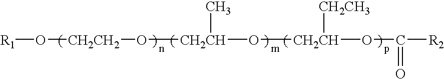
wherein R₁ is a saturated or unsaturated C₈-C₄₀ group; R₂ is a saturated or unsaturated C₈-C₂₄ group, n=1-200, m=0-200, and p=0-200.

The invention also provides a process for thickening an aqueous surfactant solution comprising adding an effective amount of the fatty acid ester of a fatty alcohol ethoxylate to an aqueous solution comprised of a surfactant.

The invention further provides a shampoo composition comprising: (a) from about 1% to about 51% by weight of a detergent selected from the group consisting of anionic surfactants, non-ionic surfactants, amphoteric surfactants and mixtures thereof, water, and from about 0.1% to about 8% by weight of a thickening agent of the formula: 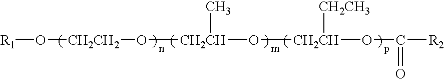
wherein R₁ is a saturated or unsaturated C₈-C₄₀ group; R₂ is a saturated or unsaturated C₈-C₂₄ group, n=1-200, m=0-200, and p=0-200.

The instant invention also provides a composition for topical application comprising from about 0.5% to about 35% by weight of an active ingredient selected from the group consisting of sunscreens, moisturizers, film formers, detergents, emulsifiers, antiseptic agents, conditioning agents, deodorant actives, pharmaceutical actives , reducing agents for permanent wave products and mixtures thereof, water and from about 0.1 to about 9% by weight of a thickening agent of the formula: 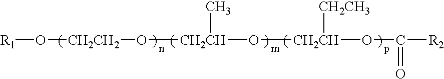
wherein R₁ is a saturated or unsaturated C₈-C₄₀ group; R₂ is a saturated or unsaturated C₈-C₂₄ group, n=1-200, m=0-200, and p=0-200.

### DESCRIPTION OF THE PREFERRED EMBODIMENTS

The thickening agents of the present invention are based upon fatty acid esters of fatty alcohol alkoxylates having the formula 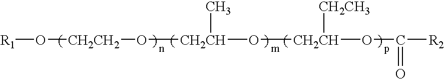
wherein R₁ is a saturated or unsaturated C₈-C₄₀ group; R₂ is a saturated or unsaturated C₈-C₂₄ group, n=1-200, m=0-200, and p=0-200.

The fatty acid esters thickening agents represented by the above formula are manufactured by reacting a fatty alcohol alkoxylate of the formula 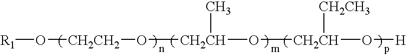
wherein R₁ is a straight or branched saturated or unsaturated C₈-C₄₀ group; n=1-200, m=0-200, and p=0-200, with a fatty acid of the formula 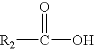
wherein R₂ is a straight or branched saturated or unsaturated C₈-C₂₄ group at a temperature range of from about 150° C. to about 250° C. in the presence or absence of a catalyst.

The fatty acids esters of alkoxylated fatty alcohols are obtained by:
(a) alkoxylating (i.e., ethoxylating a fatty alcohol) with 80 to 200 moles of alkylene oxide per mole of fatty alcohol at elevated temperatures in the presence of basic catalysts and
(b) subsequently reacting the reaction product with 1 to 1.2 moles of fatty acid per mole of the hydroxyl groups present in the fatty alcohol alkoxylate in the absence or presence of acidic catalysts.

Esters having particularly favorable thickening properties are derived from adducts of 100 to 200 moles of ethylene oxide with 1 mole of a C₈-C₄₀ alcohol which have been esterified with saturated and unsaturated fatty acids containing 8 to 24 carbon atoms.

The present invention also relates to a process for the production of esters of fatty acids with ethoxylated fatty alcohols wherein (a) fatty alcohols are ethoxylated with 80 to 200 moles of ethylene oxide per mole of fatty alcohol at elevated temperatures in the presence of basic catalysts and (b) the reaction product is subsequently reacted with 1 to 1.2 moles of fatty acid per mole of the fatty alcohol ethoxylate in the presence of acidic catalysts.

In the content of the invention, fatty alcohols which may be used as starting materials for alkoxylation and then ester derivatization according to the invention are understood to be substances which have at least 8 carbon atoms. The fatty alcohols may be alkoxylated by methods known per se. Suitable basic catalysts for this purpose are, for example, sodium hydroxide, potassium hydroxide, sodium methylate and other basic catalyst known to one of ordinary skill in the art.

The molar ratio of fatty alcohol to alkylene oxide may be in the range from 1:80 to 1:200. To obtain esters having particularly advantageous thickening properties, it has proved to be optimal to select a ratio of 1:100 to 1:200.

The alkoxylation reaction is carried out under known conditions, i.e. at elevated temperature, for example at a temperature of 120° to 190° C. and preferably 150° to 180° C., and under a pressure of 1 to 5 bar.

Typical alcohols which can be used in the alkoxylation reaction include octanol, decanol, dodecanol, lauryl alcohol, oleyl alcohol, cetyl alcohol, myristyl alcohol, stearyl alcohol, cetearyl alcohol, linoleyl alcohol and mixtures thereof are contemplated herein. In addition, mixtures of natural or synthetic fatty alcohols having fatty chain lengths of from about 8 to about 40 carbons are also useful.

After the alkoxylation, the alkoxylated fatty alcohols formed as intermediate products are subjected to esterification. The carboxylic acid component used for this purpose is selected from linear or branched saturated and unsaturated fatty acids having 8 to 24 carbon atoms. The fatty acid chain may also be substituted with hydroxyl groups.

Typical examples are lauric acid, myristic acid, palmitic acid, palmitoleic acid, stearic acid, isostearic acid, oleic acid, linoleic acid, linolenic acid, ricinoleic acid, 12-hydroxystearic acid, arachidonic acid, gadoleic acid, behenic acid, dimeric fatty acids, dimeric acids of the above fatty acids and erucic acid. Oleic acid, stearic acid and isostearic acid and technical mixtures thereof are preferred.

As usual in oleochemistry, these acids may also be present in the form of the technical cuts obtained in the pressure hydrolysis of natural fats and oils, for example palm oil, palm kernel oil, coconut oil, olive oil, sunflower oil, rapeseed oil or beef tallow. Saturated fatty acids containing 12 to 18 carbon atoms are preferred, those containing 16 to 18 carbon atoms being particularly preferred.

The esterification of the alkoxylated fatty alcohols formed as intermediate products with the fatty acids may also be carried out by methods known per se. Suitable acidic catalysts for this purpose are, for example, methanesulfonic acid, butanesulfonic acid, p-toluenesulfonic acid, naphthalenesulfonic acid, alkyl benzenesulfonic acid and/or sulfosuccinic acid.

In addition, it is advisable to carry out the esterification reaction at elevated temperatures, for example at temperatures of 140° to 275° C. and preferably 150° to 185° C. and continuously to remove the water of reaction from the equilibrium. The quantity of fatty acid used should be selected so that there are 1.0 to 1.2 and preferably 1.0 to 1.1 moles of fatty acid for every mole of the fatty alcohol alkoxylate. This ensures that the esterification of the hydroxyl groups is substantially quantitative. If desired, a residual content of free fatty acid in the end reaction product may be neutralized with alkali metal hydroxide solution.

Typical examples of the thickeners of the invention include ceteareth-150 oleate, ceteareth-150 stearate, ceteareth-150 stearate, octyldeceth-150 isostearate, octyldeceth-100 isostearate, octyldeceth-150 stearate, octyldeceth-150 stearate, octyldodeceth-100 stearate, octyldodeceth-150 stearate, octyldodeceth-150 isostearate, decyltetradeceth-150 stearate, decyltetradeceth-100 stearate, octyldodeceth-200 stearate, decyltetradeceth-100 stearate, octyldodeceth-1 50 stearate, and octyldodeceth-200 isostearate.

The esters according to the invention are used as thickeners for aqueous cosmetic compositions and aqueous surfactant solutions, to which they may be added in quantities of 0.1 to 8% by weight and preferably in quantities of 0.5 to 3% by weight, based on the solids content of the solutions.

Surfactants which may advantageously be thickened in accordance with the present invention are, for example, fatty alcohol sulfates, fatty alcohol ether sulfates, alkanesulfonates, sulfosuccinic acid monoesters and diesters, alkyl phosphates, protein fatty acid condensates, acyl isethionates, acyl taurides, soaps, alkyl ether carboxylic acids, acyl sarcosides, alkyl amidobetaines, imidazolinium betaines or sulfobetaines containing a lipophilic fatty residue with 6 to 22 carbon atoms. Surfactants of which aqueous solutions may be thickened particularly effectively by addition of the esters according to the invention are, on the one hand, fatty alcohol ether sulfates containing 12 to 18 carbon atoms in the fatty alkyl moiety and 1 to 10 ethylene oxide units in the polyether chain and also betaines.

The surfactant solutions may have a solids content of 1 to 51% by weight and preferably 5 to 30% by weight, based on the solution.

The surfactant solutions according to the invention may also contain small quantities of fragrances, dyes, opacifiers and pearlescers, antimicrobial agents, preservatives, skin-cosmetic agents, plant extracts, protein hydrolyzates, buffer substances, complexing agents and other known auxiliaries and additives typically encountered in hair shampoos, bath additives, shower bath preparations, liquid soaps, liquid skin-cleansing preparations and also in liquid laundry detergents and dishwashing detergents and liquid household cleaners.

Increasing the viscosity of aqueous solutions of the above-mentioned type with comparatively low surfactant contents is of particular interest from the point of view of practical application. In cases such as these, the addition of the esters according to the invention improves thickenability.

The surfactant compositions may also include suitable inorganic electrolyte salts such as water-soluble alkali metal, ammonium and alkaline earth metal salts, for example the fluorides, chlorides, bromides, sulfates, phosphates, nitrates, providing they are soluble in water at 20° C. in a quantity of at least 1% by weight. The chlorides or sulfates of an alkali metal, ammonium or magnesium are preferably used. Sodium chloride and magnesium chloride are particularly preferred. The inorganic electrolyte salts may be added to the aqueous surfactant solutions in concentrations of 0.1 to 5% by weight and preferably in concentrations of 0.1 to 2% by weight, based on the aqueous solution.

The thickening agents of the present invention may be employed in the formulation of a number of topical preparations, for example, shampoos, eye make-up formulations, cream rinses, lipsticks, lotions, sun screens, gels, cosmetics in general, household cleaning agents, cosmetic emulsions or cosmetic gels, hair dressing preparations, foam baths and the like. The thickening agents of the present invention are applicable to the formulation of a variety of topical preparations as noted hereinabove as falling within the scope of the present invention.

In the context of the present invention, the expression “thickened” means that the thickened surfactant solutions have a demonstrably higher viscosity than the unthickened surfactant solutions, as determined by physicochemical methods, more particularly with a Brookfield viscometer. In addition, the terms “thickener” and “thickening agent” are used synonymously.

### EXAMPLE 1

### Ethoxylation of Fatty Alcohol

The following is a general procedure for preparing the fatty alcohol alkoxylates of the invention.

(0.5 mole) of the fatty alcohol and (0.03 mole) of potassium hydroxide were introduced into a 5 liter steel autoclave in the form of a 50% by weight aqueous solution. The pressure reactor was then heated to a temperature of 110° C. over a period of 1 h and at the same time evacuated (30 mm Hg). The reaction temperature was then increased to 160° C. (55 moles or as desired depending on the degree of alkoxylation required) of an alkylene oxide were continuously introduced under pressure over a period of 2 h, an autogenous pressure of 4 to 5 bar being established. After the alkylene oxide had been added, the mixture was left to react for another 30 minutes. The reaction mixture was then cooled and freed from pressure.

### EXAMPLE 2

### General Method of Esterification

(0.15 mole) of the product of Example 1, (0.20 mole) of fatty acid (0.02 mole) of methanesulfonic acid in the form of a 70% by weight aqueous solution were introduced into a 2 liter three-necked flask equipped with a stirrer and distillation column and heated to 90° C. The reaction mixture was then heated to 160° C. and was stirred at that temperature first for 4 h under a reduced pressure of 30 mm Hg and then for another 12 h at approximately 2 mm Hg, the water of condensation being continuously distilled off from the equilibrium.

### EXAMPLE 3

Following the procedure of Example 1, the following alkoxylates were prepared:

Ceteareth-150, octyldeceth-150, octyldeceth-100, octyldodeceth-100, octyldodeceth-150, decyltetradeceth-150, decyltetradeceth-100, and octyldodeceth-200.

### EXAMPLE 4

Following the procedure of Example 2, the following fatty acid esters of fatty alcohol alkoxylates were prepared:

Ceteareth-150 oleate, ceteareth-150 stearate, ceteareth-150 stearate, octyldeceth-150 isostearate, octyldeceth-100 isostearate, octyldeceth-150 stearate, octyldeceth-150 stearate, octyldodeceth-100 stearate, octyldodeceth-150 stearate, octyldodeceth-150 isostearate, decyltetradeceth-150 stearate, decyltetradeceth-100 stearate, octyldodeceth-200 stearate, decyltetradeceth-100 stearate, octyldodeceth-150 stearate, and octyldodeceth-200 isostearate.

### EXAMPLE 5

A baby Shampoo is prepared by mixing in by weight % the following components:
Water 70.3%; PSML-80 (Polysorbate monolaurate) 5.0%, SLES (sodium laurylether sulfate) 14.0%, CAB 10.0% JR (cationic cellulose) 0.5% and Dowicil 0.2%. The solids content is about 11-12%.

### EXAMPLE 6

### Application Examples

The thickening effect of the esters according to the invention was tested in aqueous solutions of baby shampoos containing fatty alcohol ether sulfate as the surfactant. The baby shampoo composition used in the Examples of Table 1 is that of Example 5. The viscosity measurements were carried out in a Brookfield LVT viscosimeter (spindle 3n4, 0.6-12 r.p.m.). The solids content of the solution was 11-12% by weight; the temperature was 20° C. The results are set out in Table 1.

TABLE 1

| | Ratio | | | | Viscosity cPs | |
|---|---|---|---|---|---|---|
| | Ethoxylate to | | | | 1% in Baby | 2% in Baby |
| INCI Name | Acid | A.V. | S.V. | MP° C. | Shampoo | Shampoo |
| Ceteareth-150 Oleate | 90/10 | 6.8 | 20.4 | 51 | 2200 @ 6.2 | 40,000 |
| Ceteareth-150 Stearate | 91/9 | 4.8 | 18.6 | 52 | 900 @ 6.6 | 83,000 |
| Ceteareth-150 Stearate | 90/10 | 6.9 | 21.5 | 53 | 1950 @ 6.35 | 118,000 |
| Octyldeceth-150 Isostearate | 90/10 | 2.7 | 16.4 | 49 | 2300 @ 6.36 | 54,000 |
| Octyldeceth-100 Isostearate | 90/10 | 2.1 | 17.6 | 45 | 850 @ 6.4 | 44,000 |
| Octyldeceth-150 Stearate | 90/10 | 5.16 | 20.4 | 49 | 2100 @ 6.3 | 49,000 |
| Octyldeceth-150 Stearate | 90/10 | 7.3 | 18.3 | 50 | 3,250 | 99,000 |
| Octyldodeceth-100 Stearate | 90/10 | 4.9 | 19 | 51 | 2,900 | 170,000 |
| Octyldodeceth-150 Stearate | 90/10 | 6.55 | 17.7 | 48 | 4,800 | 280,000 |
| Octyldodeceth-150 Isostearate | 90/10 | 5.1 | 16.7 | 48 | 8,200 | 144,000 |
| Decyltetradeceth-150 Stearate | 91/9 | 6.3 | 14.5 | 49 | 8,600 | 380,000 |
| Decyltetradeceth-100 Stearate | 88/12 | 8.9 | 23.5 | 45 | 6,500 | 320,000 |
| Octyldodeceth-200 Stearate | 91/9 | 6.3 | 15.2 | 52 | 10,500 | 328,000 |
| Decyltetradeceth-100 Stearate | 90/10 | 6.6 | 18.5 | 45 | 9,000 | 268,000 |
| Octyldodeceth-150 Stearate | 90/10 | 8.1 | 17.3 | 49 | 10,000 | 380,000 |
| Octyldodeceth-200 Isostearate | 90/10 | 7.9 | 14.8 | 51 | 5,500 | 132,000 |
| Decyltetradeceth-200 Stearate | 90.5/9.5 | 6.2 | 18.1 | 50 | 12,600 | 410,000 |
| Decyltetradeceth-200 Isostearate | 90.5/9.5 | 6.99 | 16.8 | 54 | 2,700 | 100,000 |
| Isosteareth-150 Stearate(low diol) | 90/10 | 5.76 | 16 | 49 | 4,250 | 120,000 |
| Isosteareth-200 Stearate(low diol) | 90.5/9.5 | 7.68 | 17.79 | 56 | 4,100 | 123,000 |
| Decyltetradeceth-150 | 90/10 | 6.04 | 18.6 | 49 | 50,000 | >2,000,000 |
| Behenate(low diol) | | | | | | |
| Decyltetradeceth-150 | 90/10 | 5.22 | 20.89 | 50 | 500 | 3,500 |
| Hydroxystearate(ld) | | | | | | |
| Decyltetradeceth-150 | 90/10 | 5.94 | 17.79 | 53 | 20,400 | 1,400,000 |
| Behenate/Stearate(ld) | | | | | | |
| Decyltetradeceth-150 Oleate(low | 90/10 | 6.97 | 16.73 | 48 | 3,100 | 60,000 |
| diol) | | | | | | |
| Decyltetradeceth-150 Laurate(ld) | 90/10 | 10.22 | 20.89 | 53 | 280 | 1,000 |
| Decyltetradeceth-200 | 90/10 | 8.29 | 16.82 | 50 | 76,000 | >2,000,000 |
| Behenate/Stearate(ld) | | | | | | |
| Tetradecyloctadeceth- | 90/10 | 4.77 | 16.68 | 47 | 12,200 | 1,500,000 |
| 150Behenate/Stearate | | | | | | |
| Tetradecyloctadeceth-150 Stearate | 90/10 | 4.46 | 16.08 | 47 | 2,300 | 264,000 |
| Decyltetradeceth-200 Isostearate | 90/10 | 7.71 | 17.6 | 48 | 2,300 | 125,000 |
| Decyltetradeceth-200 Behenate (ld) | 90/10 | 6.72 | 16.72 | 49 | 65,000 | >2,000,000 |
| Decyltetradeceth-200 | 90/10 | 6.12 | 18.27 | 48 | 1,700 | 500,000 |
| Behe/Stea/Isostea(ld) | | | | | | |
| Decyltetradeceth-200 | 90/10 | 6.54 | 18.82 | 49 | 7,500 | 150,000 |
| Behe/Stea/Isostea(ld) | | | | | | |
| Decyltetradeceth-150 Isostearate | 90/10 | 9.32 | 17.39 | 47 | 1,900 | 126,000 |
| Decyltetradeceth-200 | 90/10 | 5.58 | 18.7 | 49 | 72,000 | >2,000,000 |
| Behenate/Stearate(ld) | | | | | | |
| DIMERATES | | | | | | |
| Octyldeceth-150 Dimerate | 92/8 | 4.67 | 17.71 | 47 | 1,800 | 180,000 |
| Decyltetradeceth-200 Dimerate(ld) | 90.5/9.5 | 9.79 | 18 | 49 | 770,000 | >2,000,000 |
| Octyldodeceth-200 Dimerate | 90.5/9.5 | 10.21 | 18.62 | 50 | 216,000 | >2,000,000 |
| Decyltetradeceth-100 Dimerate | 88.5/11.5 | 9.96 | 24.1 | 46 | 136,000 | >2,000,000 |
| Decyltetradeceth-200 Dimerate | 91/9 | 4.1 | 18.95 | 48 | 550,000 | >2,000,000 |
| PEG-150 Distearate | | | | | 500 | 11,800 |

Although the invention herein has been described with references to particular embodiments, it is to be understood that these embodiments are merely illustrative of the principles and application of the present invention. It is therefore to be understood that numerous modifications may be made to the embodiments and that other compositions may be devised without departing from the spirit and scope of the present invention as defined by the claims.

1. A personal care product comprising appropriate ingredients in a liquid carrier, said personal care product including an effective amount to thicken said product of a thickener of the formula 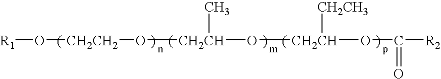
wherein R₁ is a saturated or unsaturated C₈-C₄₀ group; R₂ is a saturated or unsaturated C₈-C₂₄ group, n=1-200, m=0-200, and p=0-200.

2. The personal care product of claim 1 wherein n=1-200, m=0 and p=0.

3. The personal care product of claim 2 wherein n=50-150, m=0 and p=0.

4. The personal care product of claim 3 wherein n=150-200, m=0 and p=0.

5. A cosmetic product comprising cosmetically acceptable ingredients in a liquid carrier, said product including an effective amount to thicken said product of a thickener of the formula 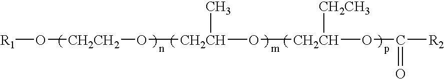
wherein R₁ is a saturated or unsaturated C₈-C₄₀ group; R₂ is a saturated or unsaturated C₈-C₂₄ group, n=1-200, m=0-200, and p=0-200.

6. The cosmetic product of claim 1 wherein said liquid carrier is an aqueous carrier.

7. The cosmetic product of claim 1 wherein said liquid carrier is a non-aqueous carrier.

8. The cosmetic product of claim 6 wherein n=100-150, m=0-200, and p=0-200.

9. The cosmetic product of claim 8 wherein said product is a shampoo.

10. The cosmetic product of claim 8 wherein said product is a shower gel.

11. The cosmetic product of claim 8 wherein said product is a topical product.

12. An aqueous composition comprising a surfactant selected from the group consisting of anionic, cationic, non-ionic, switterionic, and amphoteric surfactants and from 0.1% to 8% by weight of a thickener of the formula: 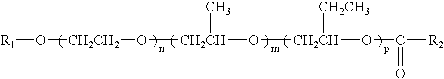
wherein R₁ is a saturated or unsaturated C₈-C₄₀ group; R₂ is a saturated or unsaturated C₈-C₂₄ group, n=1-200, m=0-200, and p=0-200.

13. A shampoo composition comprising:
(a) from about 1% to about 51% by weight of a detergent selected from the group consisting of anionic surfactants, non-ionic surfactants, amphoteric surfactants and mixtures thereof, water, and from about 0.1% to about 8% by weight of a thickening agent of the formula: 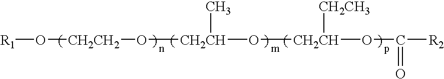
wherein R₁ is a saturated or unsaturated C₈-C₄₀ group; R₂ is a saturated or unsaturated C₈-C₂₄ group, n=1-200, m=0-200, and p=0-200.

14. The shampoo composition of claim 13, wherein said detergent is present in an amount in the range of about 5% to 18% by weight of said composition.

15. The shampoo composition of claim 13, wherein said thickening agent is present in an amount in the range of about 0.5% to 5% by weight of said composition.

16. The shampoo composition of claim 13, wherein said detergent is present in an amount of about 11% by weight of said composition and said thickening agent is present in an amount of about 1.5% by weight of said composition.

17. The shampoo composition of claim 13, wherein said detergent is selected from the group consisting of sodium lauryl sulfate, sodium laureth sulfate, ammonium lauryl sulfate, triethanolamine lauryl sulfate, polysorbate 20, lauramide DEA, sucrose monococoate, cocoamidopropyl hydroxysultaine, lauroamphodiacetate, cocamidopropyl betaine and mixtures thereof.

18. The shampoo composition of claim 13, wherein said thickening agent is selected from the group consisting of ceteareth-150 oleate, ceteareth-150 stearate, ceteareth-150 stearate, octyldeceth-150 isostearate, octyldeceth-100 isostearate, octyldeceth-150 stearate, octyldeceth-150 stearate, octyldodeceth-100 stearate, octyldodeceth-150 stearate, octyldodeceth-150 isostearate, decyltetradeceth-150 stearate, decyltetradeceth-100 stearate, octyldodeceth-200 stearate, decyltetradeceth-100 stearate, octyldodeceth-150 stearate, and octyldodeceth-200 isostearate.

19. A composition for topical application comprising from about 0.5% to about 35% by weight of an active ingredient selected from the group consisting of sunscreens, moisturizers, film formers, detergents, emulsifiers, antiseptic agents, conditioning agents, deodorant actives, pharmaceutical actives , reducing agents for permanent wave products and mixtures thereof, water and from about 0.1 to about 9% by weight of a thickening agent of the formula: 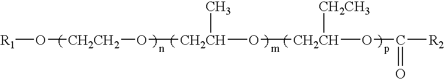
wherein R₁ is a saturated or unsaturated C₈-C₄₀ group; R₂ is a saturated or unsaturated C₈-C₂₄ group, n=1-200, m=0-200, and p=0-200.

20. The composition of claim 19, wherein said thickening agent is present in an amount in the range of about 0.1% to 8% by weight of said composition.

21. A process for thickening an aqueous surfactant solution comprising adding an effective amount of the fatty acid ester of a fatty alcohol ethoxylate to an aqueous solution comprised of a surfactant.
